(19) United States
(12) Patent Application Publication
(10) Pub. No.: US 2010/0234637 A1
(43) Pub. Date: Sep. 16, 2010

(54) INTEGRATED PROCESS TO COPRODUCE AROMATIC HYDROCARBONS AND ETHYLENE AND PROPYLENE

(76) Inventors: Howard Lam Ho FONG, Sugar Land, TX (US); Richard Dale Swain, Houston, TX (US)

Correspondence Address: SHELL OIL COMPANY, P O BOX 2463, HOUSTON, TX, 772522463, US

(21) Appl. No.: 12/724,139

(22) Filed: Mar. 15, 2010

Related U.S. Application Data
(60) Provisional application No. 61/160,391, filed on Mar. 16, 2009.

(51) Int. Cl. C07C 51/255; C07C 2/00; C07C 37/04; C07C 37/02; C07C 37/08; C07C 37/58

(52) U.S. Cl. 562/412; 585/322; 568/795; 568/796; 568/798; 568/802

(57) ABSTRACT

An integrated process for producing aromatic hydrocarbons and ethylene and/or propylene and optionally other lower olefins from low molecular weight hydrocarbons, preferably methane, which comprises: (a) contacting one or more low molecular weight alkanes, preferably methane, with a halogen, preferably bromine, under process conditions sufficient to produce a monohaloalkane, preferably monobromomethane, (b) reacting a first portion of the monohaloalkane in the presence of a coupling catalyst under process conditions sufficient to produce aromatic hydrocarbons and C₂₋₅ alkanes, (c) separating the aromatic hydrocarbons from the product mixture of step (b) to produce aromatic hydrocarbons, (d) reacting a second portion of the monohaloalkane in the presence of a coupling catalyst under process conditions sufficient to produce ethylene and/or propylene.

### CROSSREFERENCE TO RELATED APPLICATION

This application claims priority to U.S. Provisional Application Ser. No. 61/160,391 filed Mar. 16, 2009, the entire disclosure of which is hereby incorporated by reference.

### FIELD OF THE INVENTION

This invention relates to a process for the production of aromatic hydrocarbons by bromination of low molecular weight alkanes, particularly methane. More particularly, the invention relates to a process wherein aromatic hydrocarbons and ethylene and/or propylene are coproduced.

### BACKGROUND OF THE INVENTION

U.S. Pat. No. 7,244,867 describes a process for converting lower molecular weight alkanes, including methane, natural gas or ethane, propane, etc., into higher molecular weight hydrocarbons, including aromatics, by bromination to form alkyl bromides and hydrobromic acid which are then reacted over a crystalline alumino-silicate catalyst to form the higher molecular weight hydrocarbons and hydrobromic acid. Hydrobromic acid is recovered by contacting the reaction product stream with water and then converted to bromine for recycle. The higher molecular weight hydrocarbons are recovered.

In a process for producing aromatic hydrocarbons such as benzene, toluene and/or xylenes (BTX) by bromination of methane to produce monobromomethane, followed by coupling of the monobromomethane to produce aromatic hydrocarbons, the coupling reactor produces hydrogen bromide (HBr), and unintended amounts of methane, light ends (C₂₋₅ alkanes and alkenes) and heavy ends (C₉₊ aromatic hydrocarbons and possibly higher carbon number nonaromatic hydrocarbons). The basic process concept includes recycle of the light ends, possibly to a separate bromination reactor for conversion to benzene, toluene and/or xylenes (BTX), and the use of methane and heavy ends as fuel. The light ends are more easily brominated than methane and if the bromination reaction of the light ends was to be carried out in the same bromination reactor for conversion to BTX a significant portion of the light ends would be converted to higher brominated species (e.g., dibromoethane, tribromopropane, etc.). Even in a separate light ends to BTX bromination reactor, it would be impossible to effect high conversion of ethane, propane and/or butanes without over-brominating because the rate of bromination increases with increasing carbon number. Further, alkenes present in the light ends stream will be brominated to alkyl dibromides regardless of the light ends bromination configuration. The multi-brominated light end derivatives decrease bromine efficiency and are more prone to coke formation in the coupling reactor than monobromomethane. The formation of coke represents a yield loss and the necessity of frequent burning off coke increases the carbon dioxide footprint of the process and reduces process reliability. If multi-brominated light ends were separated, it would create a stream with a high concentration of compounds suspected of being considerably more toxic than monobromomethane.

It can be seen that it would be advantageous to provide an integrated process concept wherein olefins could be produced in addition to aromatic hydrocarbons, the relative amounts of olefinic and aromatic hydrocarbons produced could be easily altered, and the light ends and heavy ends could be converted into useful products. The present invention provides such an integrated process.

### SUMMARY OF THE INVENTION

The present invention provides an integrated process for producing aromatic hydrocarbons and ethylene and/or propylene, and optionally other lower olefins, from low molecular weight alkanes, preferably methane, which comprises:

(a) contacting one or more low molecular weight alkanes, preferably methane, with a halogen, preferably bromine, under process conditions sufficient to produce a monohaloalkane, preferably monobromomethane,

(b) reacting a first portion of the monohaloalkane in the presence of a coupling catalyst under process conditions sufficient to produce aromatic hydrocarbons and C₂₋₅ alkanes and optionally C₂₋₅ alkenes,

(c) separating the aromatic hydrocarbons from the product mixture of step (b) to produce aromatic hydrocarbons, and

(d) reacting a second portion of the monohaloalkane in the presence of a coupling catalyst under process conditions sufficient to produce ethylene and/or propylene.

In an embodiment, C₂₋₅ alkanes and alkenes may also be produced in step (b). After C₂ removal, the C₃₊ stream may be liquefied after optional hydrotreating to produce an LPG stream. In another embodiment, aromatic C₉₊ hydrocarbons produced in step (b) may be separated and reproportionated with toluene to produce xylenes and/or hydrodealkylated to produce benzene, toluene and/or xylenes.

In another embodiment, C₄₊ alkanes and alkenes may be separated from the other alkanes and alkenes and, together with alkylbromide byproducts produced in step (d), may be recycled to step (b).

In another embodiment, part of the ethylene and/or propylene produced in step (d) may optionally be recycled to step (b) to achieve the desired aromatics and lower olefins balance and the desired ethylene/propylene production ratio.

In another embodiment, at least some unconverted methane and/or at least some of any produced methane is recovered and recycled to step (a). In another embodiment, ethane and/or propane is produced in step (b) and/or (c) and/or (d) and at least some of the ethane and/or propane is recycled to step (a). In another embodiment, multi-brominated methane species from step (a) are separated from the monobromomethane prior to steps (b) and (d) and are recycled to step (a).

In an embodiment, hydrogen bromide is produced in the process and at least some of the hydrogen bromide (HBr) so produced may be converted to bromine which may be recycled to step (a). In another embodiment, HBr is produced in the bromination step and may be removed prior to the coupling step. In another embodiment, at least some HBr is present in the C₂₋₅ alkanes stream and may be removed therefrom prior to liquefying it to an LPG stream.

### BRIEF DESCRIPTION OF THE DRAWING

FIG. 1 is a flow diagram illustrating the process of the present invention.

### DETAILED DESCRIPTION OF THE INVENTION

The present invention provides a process for the production of aromatic compounds and ethylene and/or propylene from low molecular weight alkanes, primarily methane. Other alkanes, such as ethane, propane, butane, and pentane, may be mixed in with the methane. First, at least one low molecular weight alkane, preferably methane, is halogenated by reacting it with a halogen, preferably bromine.

The monohaloalkane, preferably monobromomethane, which is produced thereby may be contacted with a suitable coupling catalyst which causes the monohaloalkane to react with itself to produce higher molecular weight hydrocarbons such as aromatics and a mixture of intermediate range molecular weight alkanes and likely some alkenes, particularly those having from 2 to 5 carbon atoms. A small amount of methane may also be produced. The aromatic compounds, such as benzene, toluene and xylenes, may be separated from the methane and C₂₋₅ alkanes and alkenes. After an optional clean-up step to remove residual hydrogen bromide, the C₃₋₅ alkanes or a portion thereof may then be hydrotreated and liquefied to an LPG stream. It is possible that C₂₋₃ and/or C₄₊ olefins and/or diolefins may be produced. These may be recycled to coupling. Higher molecular weight aromatic hydrocarbons may also be produced in the coupling step, such as those containing nine or more carbon atoms. These C₉₊ hydrocarbons may be processed as described below and converted into more desirable aromatic hydrocarbons such as benzene, toluene and/or xylenes.

The hydrocarbon feed may be comprised of a low molecular weight alkane. Low molecular weight alkanes include methane, ethane and propane, as well as butane and pentane. The preferred feed is natural gas which is comprised of methane and often contains smaller amounts of ethane, propane and other hydrocarbons. The most preferred feed is methane.

Higher molecular weight hydrocarbons are defined herein as those hydrocarbons having a greater number of carbon atoms than the components of the lower molecular weight hydrocarbon feedstock. Higher molecular weight hydrocarbons include aromatic hydrocarbons, especially benzene, toluene and xylenes (hereinafter referred to as “BTX”).

Representative halogens include bromine and chlorine. It is also contemplated that fluorine and iodine may be used but not necessarily with equivalent results. Some of the problems associated with fluorine possibly may be addressed by using dilute streams of fluorine. It is expected that more vigorous reaction conditions will be required for alkyl fluorides to couple and form higher molecular weight hydrocarbons. Similarly, problems associated with iodine (such as the endothermic nature of some iodine reactions) may likely be addressed by carrying out the halogenation and/or coupling reactions at higher temperatures and/or pressures. The use of bromine or chlorine is preferred and the use of bromine is most preferred. While the following description may only refer to bromine, bromination and/or bromomethanes, the description is applicable to the use of other halogens and halomethanes as well.

Bromination of the methane (methane will be used in the following description but other alkanes may be used or may be present as discussed above) may be carried out in an open pipe, a fixed bed reactor, a tube-and-shell reactor or another suitable reactor, preferably at a temperature and pressure where the bromination products and reactants are gases. Fast mixing between bromine and methane is preferred to help prevent over-bromination and coking. For example, the reaction pressure may be from about 100 to about 5000 kPa and the temperature may be from about 150 to about 600° C., more preferably from about 350 to about 550° C. and even more preferably from about 450 to about 525° C. Higher temperatures tend to favor coke formation and lower temperatures require larger reactors. Methane bromination may be initiated using heat or light with thermal means being preferred.

A halogenation catalyst may also be used. In an embodiment, the reactor may contain a halogenation catalyst such as a zeolite, amorphous alumino-silicate, acidic zirconia, tungensteastes, solid phosphoric acids, metal oxides, mixed metal oxides, metal halides, mixed metal halides (the metal in such cases being for example nickel, copper, cerium, cobalt, etc.) and/or other catalysts as described in U.S. Pat. Nos. 3,935,289 and 4,971,664, each of which is herein incorporated by reference in its entirety. Specific catalysts include a metal bromide (for example, sodium bromide, potassium bromide, copper bromide, nickel bromide, magnesium bromide and calcium bromide), a metal oxide (for example, silicon dioxide, zirconium dioxide and aluminum trioxide) or metal (for example, platinum, palladium, ruthenium, iridium, or rhodium) to help generate the desired brominated methane.

The bromination reaction product comprises monobromomethane, HBr and also small amounts of dibromomethane and tribromomethane.

If desired, the HBr may be removed prior to coupling. The presence of large concentrations of the polybrominated species in the feed to the coupling reactor may decrease bromine efficiency and result in an undesirable increase in coke formation. In many applications, such as the production of aromatics and light olefins, it is desirable to feed only monobromomethane to the coupling reactor to improve the conversion to the final higher molecular weight hydrocarbon products. In an embodiment of the invention, a separation step is added after the halogenation reactor in which the monobromomethane is separated from the other bromomethanes.

The di- and tribromomethane species may be recycled to the bromination reactor. One separation method is described in U.S. Published Patent Application No. 2007/02388909, which is herein incorporated by reference in its entirety. Preferably, the separation is carried out by distillation. The di- and tribromomethanes are higher boiling than the monobromomethane, unreacted methane and HBr. HBR is also made in the bromination reaction:

CH₄+Br₂→CH₃Br+HBr

In a preferred embodiment, the polybromomethanes may be recycled to the halogenation reaction and preferably reproportionated with methane to convert them to monobromomethane. The polybromomethanes contain two or more bromine atoms per molecule. Reproportionation may be accomplished according to U.S. Published Patent Application 2007/0238909 which is herein incorporated by reference in its entirety. Reactive reproportionation is accomplished by allowing the methane feedstock and any recycled alkanes to react with the polybrominated methane species from the halogenation reactor, preferably in the substantial absence of molecular halogen. Reproportionation may be carried out in a separate reactor or in a region of the halogenation reactor.

The bromination and coupling reactions may be carried out in separate reactors or the process may be carried out in an integrated reactor, for example, in a zone reactor as described in U.S. Pat. No. 6,525,230 which is herein incorporated by reference in its entirety. In this case, halogenation of methane may occur within one zone of the reactor and may be followed by a coupling step in which the liberated hydrobromic acid may be adsorbed within the material that catalyzes condensation of the halogenated hydrocarbon. Hydrocarbon coupling may take place within this zone of the reactor and may yield the product higher molecular weight hydrocarbons including aromatic hydrocarbons. It is preferred that separate reactors be used for bromination and coupling because operating conditions may be optimized for the individual steps and this allows for the possibility of removing polybrominated-methane before the coupling step.

A first portion of the monohaloalkane, preferably monobromomethane, which is produced thereby may be contacted with a suitable coupling catalyst under process conditions which cause the monohaloalkane to react with itself to produce higher molecular weight hydrocarbons such as aromatics and a mixture of intermediate range molecular weight alkanes and likely some alkenes, particularly those having from 3 to 5 carbon atoms. A small amount of ethane/ethylene and methane may also be produced. The aromatic compounds, such as benzene, toluene and xylenes, may be separated from the methane/ethane/ethylene and C₃₋₅ alkanes and alkenes. After an optional clean-up step to remove residual hydrogen bromide, the C₃₋₅ alkanes and alkenes or a portion thereof may then be hydrotreated and liquefied into a LPG stream. Higher molecular weight aromatic hydrocarbons may also be produced in the coupling step, such as those containing nine or more carbon atoms. These C₉₊ hydrocarbons may be processed as described below and converted into more desirable aromatic hydrocarbons such as benzene, toluene and/or xylenes. In a preferred embodiment, the first coupling reaction may be carried out such that the production of aromatic hydrocarbons, specifically BTX, is maximized. The production of aromatic hydrocarbons may be achieved by the use of a suitable coupling catalyst under suitable operating conditions.

Coupling of monobromomethane to produce aromatic hydrocarbons may be carried out in a fixed bed, fluidized bed or other suitable reactor. The temperature may range from about 150 to about 600° C., preferably from about 300 to about 550° C., most preferably from about 350 to about 475° C., and the pressure may range from about 10 to about 3500 kPa absolute, preferably about 100 to about 2500 kPa absolute. In general, a relatively long residence time favors conversion of reactants to products as well as product selectivity to BTX, while a short residence time means higher throughput and possibly improved economics. It is possible to change product selectivity by changing the catalyst, altering the reaction temperature, pressure and/or altering the residence time in the reactor. Low molecular weight alkanes may also exit the coupling reactor. These low molecular weight alkanes may be comprised of ethane and propane but may also include methane and a small amount of C₄₋₅ alkanes and smaller amounts of alkenes. Some of these may be recycled to the bromination reactor but preferably the low molecular weight alkanes may be directed to the liquefaction step.

Preferred coupling catalysts for use in the present invention are described in U.S. Patent Application No. 2007/0238909 and U.S. Pat. No. 7,244,867, each of which is herein incorporated by reference in its entirety. A metal-oxygen cataloreactant may also be used to facilitate the coupling reaction. The term “metal-oxygen cataloreactant” is used herein to a cataloreactant material containing both metal and oxygen. Such cataloreactants are described in detail in U.S. Published Patent Application Nos. 2005/0038310 and 2005/0171393 which are herein incorporated by reference in their entirety. Examples of metal-oxygen cataloreactants given therein include zeolites, doped zeolites, metal oxides, metal oxide-impregnated zeolites and mixtures thereof. Nonlimiting examples of dopants include alkaline earth metals, such as calcium, magnesium, manganese and barium and their oxides and/or hydroxides.

A second portion of the monohaloalkane, preferably monobromomethane, which is produced in the halogenation step may be contacted with a suitable coupling catalyst under process conditions which cause the monohaloalkane to react with itself to produce primarily ethylene and/or propylene. The reaction conditions for producing primarily ethylene and/or propylene may be different from the reaction conditions for producing primarily aromatic hydrocarbons.

Suitable reaction conditions for the olefin coupling reaction are described in U.S. Published Patent Application Nos. 2007/0238909, 2005/0038310 and 2005/0171393, all of which are herein incorporated by reference in their entirety. Temperatures of from about 300 to about 450° C. are preferred to produce lower molecular weight olefins but the temperature may range from about 150 to about 600° C. Lower pressures favor less carbon-carbon coupling and thus lower molecular weight products. Pressures of from about 10 to about 3500 kPa may be used.

Coupling catalysts which may be used for producing primarily ethylene and/or propylene may be different from the catalysts which may be used for producing primarily aromatic hydrocarbons. The preferred catalysts are described in detail in U.S. Published Patent Application Nos. 2007/0238909, 2005/0038310 and 2005/0171393 which are herein incorporated by reference in their entirety. The catalyst may be a metal-oxygen cataloreactant which facilitates carbon-carbon coupling, i.e., hydrocarbon oligomerization. The term “metal-oxygen cataloreactant” is used herein to a cataloreactant material containing both metal and oxygen. Nonlimiting examples of metal-oxygen cataloreactants given therein include zeolites, doped zeolites, metal oxides, metal oxide-impregnated zeolites and mixtures thereof. Nonlimiting examples of dopants include alkaline earth metals, such as calcium, magnesium, manganese and barium and their oxides and/or hydroxides.

As described in U.S. Published Patent Application Nos. 2007/0238909, 2005/0038310 and 2005/0171393, shifting the properties of the zeolite or zeolite component of a zeolite/metal oxide composite is expected to shift product distribution of this reaction. Pore size and acidity are particularly expected to be important. Acidity may be used to control chain length and functionality and pore size may control chain length and functionality. The ratio of silicon to aluminum (Si:Al) in the zeolite may influence the acidity of the zeolite and thus the product distribution. In particular, the Si:Al ratio may influence the relative amounts of olefins and other hydrocarbons in the product. Specifically, ZSM-5 is likely to produce a significant yield of ethylene and propylene.

In one embodiment of the invention, a metal oxide/zeolite composite is prepared by mixing a zeolite with a metal nitrate (e.g., an alkaline-earth metal oxide, such as calcium nitrate, or other metal nitrate such as manganese nitrate) or hydrated species thereof, and then calcining this mixture to release nitrogen oxides and retain the metal oxide-impregnated zeolite. The metal-oxygen cataloreactant may be regenerated by treatment with air or oxygen, typically at a temperature of from about 200 to about 900° C. This converts metal halide species into metal-oxygen species.

Control of the feed composition may allow control of the product distribution. For example, monobromomethane compared to dibromomethane may be expected to drive the production of olefins, including ethylene and/or propylene. In U.S. Published Patent Application No. 2005/0171393 brominated methane was contacted with a catalyst incorporating a ZSM-5-type zeolite (Zeolyst CBV 8014, Si/Al ratio=80:1) and CaNO₃ nonahydrate at 400° C. and a product mixture containing 10% ethylene, 31% propylene, 3% propane, and 21% butanes/butenes was produced. In another example, methane was contacted with a catalyst incorporating a mordenite (Zeolyst CBV 21A) doped with both Ca and Mg under the same conditions and a product mixture containing 30% ethylene, 5% ethane, 10% propylene, 3% propane, and 5% butanes/butenes was produced.

Hydrogen bromide may also be produced along with monobromomethane in the bromination reactor. The hydrogen bromide may be carried over to the coupling reactor(s) or, if desired, may be separated before coupling. The products of the olefin coupling reaction may include a small amount of methane, C₂ alkenes and alkanes, C₃-C₅ alkenes and alkanes, alkyl-bromides resulting from hydrobromination of product alkenes with hydrogen bromide (e.g., monobromomethane, monobromomethane, monobromopropane, monobromobutane, etc.), BTX, a small amount of C₉₊ aromatics, and hydrogen bromide. In a preferred embodiment, the hydrogen bromide may be separated from the higher molecular weight hydrocarbon product by distillation.

The coupling reaction product higher molecular hydrocarbons and hydrogen bromide may be sent to an absorption column wherein the hydrogen bromide may be absorbed in water using a packed column or other contacting device. Input water in the product stream may be contacted either in co-current or countercurrent flow with countercurrent flow preferred for its improved efficiency. One method for removing the hydrogen bromide from the higher molecular weight hydrocarbon reaction product is described in U.S. Pat. No. 7,244,867 which is herein incorporated by reference in its entirety. HBr present in the C₂₋₅ alkanes and alkenes stream or the product stream from the bromination reactor may also be removed therefrom by this method.

In an embodiment, the hydrogen bromide is recovered by displacement as a gas from its aqueous solution in the presence of an electrolyte that shares a common ion or an ion that has a higher hydration energy than hydrogen bromide. Also aqueous solutions of metal bromides such as calcium bromide, magnesium bromide, sodium bromide, potassium bromide, etc. may be used as extractive agents.

In another embodiment, catalytic halogen generation is carried out by reacting hydrogen bromide and molecular oxygen over a suitable catalyst. The oxygen source may be air, pure oxygen or enriched air. A number of materials have been identified as halogen generation catalysts. It is possible to use oxides, halides, and/or oxyhalides of one or more metals, such as magnesium, calcium, barium, chromium, manganese, iron, cobalt, nickel, copper, zinc, etc. After the HBr is separated from the hydrocarbon products, it may be reacted to produce bromine for recycle to the bromination step. Catalysts and methods for regeneration of the bromine are described in detail in U.S. Published Application 2007/0238909 which is herein incorporated by reference in its entirety. Recovery of bromine is also described therein.

In addition to the BTX and the hydrogen bromide, other materials may exit from the aromatic hydrocarbon coupling reactor. These include methane, light ends (C₂₋₅ alkanes and alkenes) and heavy ends (aromatic C₉₊ hydrocarbons and a small amount of nonaromatic C₆₊ hydrocarbons, usually less than 1%). The methane may be separated from these other materials (e.g., by distillation) and recycled to the bromination reactor. The C₃₋₅ alkanes, and optionally the alkenes, may be separated from the other materials, hydrotreated and liquefied into a LPG stream. The C₃₋₅ alkanes and alkenes stream may contain some HBr which may be removed prior to hydrotreating/liquefaction. The C₉₊ aromatic hydrocarbons may be converted to xylenes by transalkylation with toluene, hydrodealkylated to BTX or they may be upgraded by a combination of these two steps.

In addition to the C₂/C₃ alkenes and alkanes and the hydrogen bromide, other materials may exit from the olefin coupling reactor. These include a small amount of methane, C₄/C₅ alkenes and alkanes, C₂₊ alkylbromides, BTX, and C₉₊ aromatics. The C₄₊ hydrocarbons and C₂₊ alkybromides are separated from the C₁-C₃ hydrocarbon and recycled to the aromatics coupling reaction.

Depending upon the specific combination of process steps described above which are utilized, the present invention may provide some or all of the following advantages:

(1) By not recycling C₂₋₅ hydrocarbons to the methane bromination step, the safety issues associated with handling a concentrated stream of multibrominated hydrocarbons are avoided.

(2) A separate bromination/reproportionation reactor for bromination of a C₂₋₅ hydrocarbon recycle stream is eliminated.

(3) Coking in the coupling reactor may be reduced significantly and the amount of polybrominated methane which must be removed between the bromination and coupling steps may also be reduced.

(4) Generation of ethylene and/or propylene as products, and optionally other lower olefins, in addition to aromatic hydrocarbons delivers a higher value-added product mix.

(5) It is difficult to separate the alkylbromides co-produced in the olefin coupling reaction from the C₄⁺ hydrocarbons by distillation. By recycling the entire stream to the aromatics coupling reaction step to produce more aromatics, such a separation step is avoided.

(6) By varying the split of the monobromomethane going to the aromatics and olefin coupling reaction, respectively, BTX and lower olefin (LO) production flexibility is achieved.

(7) By optionally recycling part of the ethylene and/or propylene to the aromatics coupling reaction, the desired ratio of ethylene to propylene may be produced.

(8) By co-producing the desired amount of benzene and ethylene and propylene, ethylbenzene/styrene and cumene/phenol may be optionally produced on site without first processing the C₂ and C₃ streams through the expensive C₂ and C₃ splitters, respectively.

Heat from the conversion of methane to aromatics and light hydrocarbons, including heat generated in the generation of bromine, may be used in the process to supply energy required in heating the feed streams for the bromination, reproportionation and/or coupling reactions and for heat required in any of the fractionation operations. At least part of the energy released in the conversion of hydrogen bromide to bromine may be recovered and utilized in steps (a)-(d) or any combination thereof and optionally in upstream (including but not limited to gas feedstock processing) and/or downstream processing (including, but not limited to light olefin and BTX conversion and purification, disproportion reactions, aromatic C₉₊ hydrocarbon transalkylation reactions, isomerization reactions, and conversion of ethylene and propylene and BTX to downstream products).

The ethylene, propylene and other olefins which may be produced may then be used to produce many commercial chemical products, for example, polyethylene, polypropylene, ethylene glycol, ethylene oxide, alpha olefins, detergent-range alcohols, propylene glycol, propylene oxide, acrylic acid, ethanol, n-butanol, 2-ethyl-hexanol, etc.

Phenol can be made from the partial oxidation of benzene or benzoic acid, by the cumene process or by the Raschig process. It can also be found as a product of coal oxidation.

The cumene process is an industrial process for developing phenol and acetone from benzene and propylene in which cumene is the intermediate material during the process. This process converts two relatively inexpensive starting materials, benzene and propylene, into two more valuable ones, phenol and acetone. Other reactants required are oxygen from air and small amounts of a radical initiator. Most of the worldwide production of phenol and acetone is now based on this method.

Cumene is the common name for isopropylbenzene. Nearly all the cumene that is produced as a pure compound on an industrial scale is converted to cumene hydroperoxide which is an intermediate in the synthesis of other industrially important chemicals such as phenol and acetone.

Cumene was for many years been produced commercially by the alkylation of benzene with propylene over a Friedel-Crafts catalyst, particularly solid phosphoric acid or aluminum chloride such as described in U.S. Pat. No. 4,343,957. More recently, however, zeolite-based catalyst systems have been found to be more active and selective for propylation of benzene to cumene. It is known that aromatic hydrocarbons can be alkylated in the presence of acid-treated zeolite. U.S. Pat. No. 4,393,262 (1983) teaches that cumene is prepared by the alkylation of benzene with propylene in the presence of a specified zeolite catalyst. U.S. Pat. No. 4,992,606 describes the use of MCM-22 zeolite in the alkylation of benzene with propylene. Other methods are described in U.S. Pat. Nos. 4,441,990, 5,055,627, 6,525,236 and 6,888,037. All of these patents are herein incorporated by reference in their entirety.

In one embodiment, cumene may be produced by contacting benzene with propylene in a distillation column reactor containing a fixed bed acidic catalytic distillation structure comprising a molecular sieve in a distillation reaction zone thereby catalytically reacting the benzene and propylene to produce an alkylated benzene product including cumene. Cumene may be produced in the catalyst bed under 0.25 to 50 atmospheres of pressure and at temperatures in the range of 50° C. to 500° C., using as the catalyst a mole sieve characterized as acidic. Propylene may be fed to the catalyst bed while benzene may be conveniently added through a reflux to result in a molar excess present in the reactor to that required to react with propylene, thereby reacting substantially all of the propylene and recovering benzene as the principal overhead and cumene and diisopropyl benzene in the bottoms. Concurrently, in the fixed bed the resultant alkylated benzene product is fractionated from the unreacted materials and cumene is separated from the alkylated benzene product (preferably by fractional distillation).

The principal alkylated benzene product is cumene. In addition there may be other alkylated products including di and tri isopropyl benzene, n-propyl benzene, ethyl benzene, toluene, diethyl benzene and di-n-propyl benzene, which are believed to be disproportion and isomerization products of cumene. In a preferred process the residual alkylated products remaining after cumene separation may be passed to a transalkylation reactor operated under conditions to transalkylate polyalkylated benzene, e.g., diisopropyl benzene and triisopropyl benzene, to cumene which may be separated from the other materials in the transalkylation product stream and may be combined with the cumene from the first separation.

Cumene may be oxidized in slightly basic conditions in presence of a radical initiator which removes the tertiary benzylic hydrogen from cumene and hence forms a cumene radical. This cumene radical then bonds with an oxygen molecule to give the cumene hydroperoxide radical, which in turn forms cumene hydroperoxide (C₆H₅C(CH₃)₂—O—O—H) by abstracting benzylic hydrogen from another cumene molecule. This cumene hydroperoxide converts into cumene radicals and feeds back into subsequent chain formations of cumene hydroperoxides. A pressure of at least about 5 atm may be used to ensure that the unstable peroxide is kept in liquid state.

For example, cumene hydroperoxide may be made according to the process described in U.S. Pat. No. 7,141,703, which is herein incorporated by reference in its entirety. The process comprises providing an oxidation feed consisting essentially of an organic phase. The oxidation feed comprises one or more alkylbenzenes such as cumene and a quantity of neutralizing base having a pH of from about 8 to about 12.5 in 1 to 10 wt. % aqueous solution. The quantity of neutralizing base is effective to neutralize at least a portion of acids formed during the oxidation. The oxidation feed comprises up to an amount of water effective to increase neutralization of acids formed during the oxidation without forming a separate aqueous phase. The oxidation feed is exposed to oxidation conditions effective to produce an oxidation product stream comprising one or more product hydroperoxides.

Cumene hydroperoxide may then be hydrolyzed in an acidic medium to give phenol and acetone.

Additional technologies such as benzene sulfonation/hydrolysis and benzene chlorination/hydrolysis processes may also be used to convert the benzene into phenol, although currently they are not as economically competitive as the cumene process.

The direct oxidation of benzene using air or oxygen is another way in which benzene may be converted into phenol according to the present invention. It does not require reaction with propylene. For example, U.S. Pat. No. 4,992,600, which is herein incorporated by reference in its entirety, describes a process for the oxidation of benzene to phenol which comprises contacting and thereby reacting benzene and oxygen with a (poly)metal salt of a dihydrodihydroxyanthracene(poly)sulfonate having at least one sulfonate moiety on the 2, 3, 6 or 7 position(s) and which salt is dissolved in water, optionally in the presence of an oxidation catalyst, and subsequently separating from the reaction product phenol and the corresponding (poly)metal salt of anthraquinone-(poly)sulfonate. The by-product anthraquinone salt is suitably recycled to the benzene oxidation step by hydrogenating the anthraquinone salt, preferably dissolved in water, to the dihydrodihydroxyanthracene salt by contacting it with hydrogen in the presence of a hydrogenation catalyst.

Additionally, U.S. Pat. No. 6,900,358, which is herein incorporated by reference in its entirety, describes a process for the oxidation of benzene to phenol which comprises continuously contacting, in a distillation column reactor comprising a reaction zone and a distillation zone, benzene with a zeolite catalyst and an oxidant at a temperature in the range of from above 100° C. to 270° C. thereby producing a hydroxylated product, wherein at least a portion of the benzene being in a liquid phase; continuously separating the hydroxylated product from the un-reacted benzene in the distillation zone under conditions effective to vaporize said un-reacted benzene and maintain the hydroxylated product in a liquid phase; and recovering the hydroxylated product from the distillation column reactor.

The integrated process of this invention may also include the reaction of benzene with olefins such as ethylene. The ethylene may be produced separately in an ethane dehydrogenation unit or may come from olefin cracker process vent streams or other sources.

Ethylbenzene is an organic chemical compound which is an aromatic hydrocarbon. Its major use is in the petrochemical industry as an intermediate compound for the production of styrene, which in turn is used for making polystyrene, a commonly used plastic material. Although often present in small amounts in crude oil, ethylbenzene is produced in bulk quantities by combining the petrochemicals benzene and ethylene in an acidically-catalyzed chemical reaction. Catalytic dehydrogenation of the ethylbenzene then gives hydrogen gas and styrene, which is vinylbenzene. Ethylbenzene is also an ingredient in some paints.

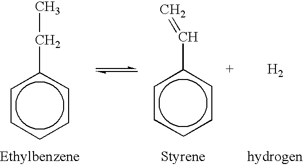

Ethylbenzene may, for example, be produced according to the process of U.S. Pat. No. 5,243,116, which is herein incorporated by reference in its entirety. The process comprises alkylating benzene by contacting the benzene with ethylene in the presence of a catalyst consisting essentially of an acidic mordenite zeolite having a silica/alumina molar ratio of at least 30:1 and a crystalline structure which is determined by X-ray diffraction to be a matrix of Cmcm symmetry having dispersed therein domains of Cmmm symmetry and having a Symmetry Index of at least about 1.

Another process for producing ethylbenzene from benzene is described in U.S. Pat. No. 5,877,370, which is herein incorporated by reference in its entirety. The process comprises passing benzene, ethylene, and a diluent comprising at least one phenyl group and at least one ethyl group to an alkylation zone; reacting the benzene and the ethylene in the alkylation zone in the presence of zeolite beta to alkylate the benzene to form ethylbenzene; and withdrawing from the alkylation zone a product comprising ethylbenzene.

Styrene may then be produced by dehydrogenating the ethylbenzene. One process for producing styrene is described in U.S. Pat. No. 4,857,498, which is herein incorporated by reference in its entirety. Another process for producing styrene is described in U.S. Pat. No. 7,276,636, which is herein incorporated by reference in its entirety. This process for producing styrene comprises: a) reacting benzene and a polyethylbenzene in a transalkylation reactor to form ethylbenzene; b) dehydrogenating ethylbenzene in a dehydrogenation reactor to form styrene; c) withdrawing a dehydrogenation reactor effluent comprising styrene from the dehydrogenation reactor, and passing at least a portion of the dehydrogenation reactor effluent to a dehydrogenation separation section; d) recovering styrene from the dehydrogenation separation section; e) introducing a first inhibitor element component to the dehydrogenation separation section; f) recovering from the dehydrogenation separation section a recycle stream comprising a second inhibitor element component; and g) passing at least 33% of the second inhibitor element component recovered in f) to the transalkylation reactor.

Terephthalic acid is an important intermediate for the production of polyester polymers which are used typically for fibre production and in the manufacture of bottles. Current state-of-the-art technology for the manufacture of terephthalic acid involves the liquid phase oxidation of paraxylene feedstock using molecular oxygen, or a gas containing molecular oxygen such as air, in a solvent comprising lower C₂ to C₆ aliphatic monocarboxylic acid, usually acetic acid, in the presence of a dissolved heavy metal catalyst system such as a cobalt-containing catalyst comprising a cobalt compound, a manganese compound and a promoter such as bromine or a bromine-yielding compound.

The reaction may be carried out in at least one stirred vessel under elevated temperature and pressure conditions, typically 150 to 250° C. and 600 to 3000 kPa respectively, with air being sparged into the reaction mixture. The oxidation of para-xylene may be performed in a fractionating zone, preferably a catalytic liquid phase oxidation zone the upper portion of which is directly connected a distillation tower. This method typically produces terephthalic acid in high yield, e.g. at least 95%. Isothermal reaction conditions may be maintained in the oxidation vessel by allowing evaporation of the solvent, together with water produced in the reaction, the resulting vapour being condensed and returned to the reactor vessel as reflux.

In the conventional production of terephthalic acid, because terephthalic acid is only sparingly soluble in the solvent, a substantial proportion the product precipitates in the course of the reaction and as a result impurities such as 4-carboxybenzaldehyde (4-CBA) and colour bodies co-precipitate with the terephthalic acid to produce a crude product which, to meet the requirements of many polyester producers, has to be purified to reduce its impurity content. In one purification process, the crude product is dissolved in water and, under elevated temperature and pressure conditions, is contacted with hydrogen in the presence of a hydrogenation catalyst, the purified terephthalic acid thereafter being recovered by crystallisation and solids-liquid separation techniques.

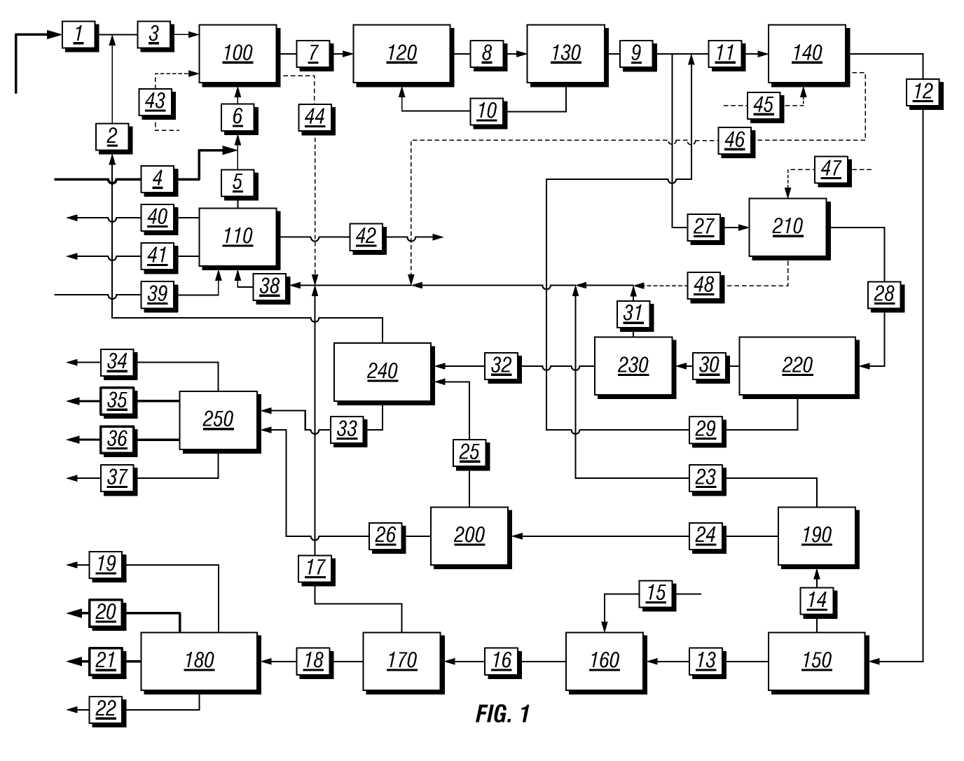

One embodiment of the invention is illustrated in FIG. 1. Methane derived from natural gas purification is delivered through line 1 at 30 barg (3000 kPag) and ambient temperature. The methane stream is combined with recycle methane stream 2, heated to 450° C., and the combined methane feed stream 3 is fed to the bromination reactor 100. Bromine liquid is pumped from storage in line 5, combined with a small make-up bromine stream 4, vaporized and heated to 250° C., and the combined bromine stream 6 is fed in a staged manner into to the bromination reactor 100.

In the bromination reactor 100, bromine reacts adiabatically with methane to form methyl bromide, methyl dibromide, methyl tribromide, and hydrogen bromide. In this example, the reactor does not utilize a catalyst. During normal operation, a small amount of coke is produced. The bromination reactor 100 is comprised of at least 2 parallel reactor trains to allow for one train to be decoked while the other train(s) remains in normal operation. Heated air in line 43 is used to periodically decoke the Bromination Reactor. Reactor effluent gas from the decoking operation is routed through line 44 to the bromine generation reactor 110 (described below).

A gas mixture containing methyl bromides, hydrogen bromide and unreacted methane, exits the bromination reactor 100 through line 7 at 510° C. and 30 barg (3000 kPag) and enters the reproportionation reactor 120. The reproportionation product gas stream 8 is cooled and fractionated in a conventional distillation column 130 to separate polybromides from the other reproportionation products. Polybrominated hydrocarbons (stream 10), recovered from distillation column 130, are recycled to the reproportionation reactor 120 where di- and tri-substituted methyl bromide and other polybrominated hydrocarbons react adiabatically with unreacted methane to form methyl bromide. In this example, the reproportionation reactor 120 does not utilize a catalyst.

The remaining components of the reproportionation product stream 8 (primarily methyl bromide, hydrogen bromide, and unreacted methane) are recovered as a separate stream 9. A portion of stream 9 is combined with stream 29 (comprised of recovered C₄ₛ, C₅ₛ, and brominated ethane and propane), vaporized, heated to 400° C., and fed to the BTX coupling reactor 140 as stream 11.

In the BTX coupling reactor 140, methyl bromide reacts adiabatically over a manganese-based catalyst at a temperature of 425° C. and 25 barg (2500 kPag) to produce a mixture of compounds comprised predominately of benzene, toluene, xylenes, ethane, propane, butane, and pentanes. The BTX coupling reactor 140 is comprised of multiple fixed bed catalytic reactors operating on a reaction/regeneration cycle. During the reaction phase, methyl bromide reacts to form mixed products. At the same time, coke is formed and gradually deactivates the catalyst.

During the regeneration phase of a reactor, reactor feed is redirected to reactors in the reaction phase of the cycle. Heated air stream 45 is utilized to burn coke and regenerate the catalyst. Reactor effluent gas stream 46 from the decoking operation is routed to the bromine generation reactor to recover bromine.

Product gas from the BTX coupling reactor 140 is directed through line 12 and cooled and fractionated in conventional distillation column 150 to produce two streams. The higher boiling stream, 13, is comprised primarily of benzene, toluene, and xylenes. The lower boiling stream 14 is comprised primarily of methane, ethane, propane, butanes, pentanes, and hydrogen bromide.

The higher boiling stream 13 from distillation column 160 is heated and routed to product cleanup reactor 160. Hydrogen from line 15 is added to this adiabatic trickle phase reactor, which uses a palladium-based catalyst to convert residual hydrocarbon bromides to the equivalent alkanes and hydrogen bromide.

Stream 16 exiting the product cleanup reactor 160 is fractionated in conventional distillation column 170 to recover bromine-free stream 18 which is comprised primarily of benzene, toluene, and xylenes (BTX) and a lights stream 18 comprised primarily of hydrogen bromide, unreacted hydrogen, and light hydrocarbons (produced by de-halogenation in product cleanup reactor 160). The lights stream 17 is routed to the bromine generator 110 to recover bromine.

BTX stream 18 is fed to a conventional integrated paraxylene unit comprised of benzene separation, paraxylene purification (e.g. via adsorption or crystallization), and transalkylation of C₈ₛ/C₉ₛ/C₁₀ₛ to maximize paraxylene production. Streams produced by the paraxylene unit are a light ends fuel stream 19, a product benzene stream 20, a product paraxylene stream 21, and a heavy ends fuel stream 22.

Stream 14 from distillation column 150 (comprised of hydrogen bromide and C₁-C₅ alkanes) is sent to an aqueous HBr recovery system 190 that produces an organic-free HBr stream 23, and an HBR-free stream 24. HBr recovery system 190 is comprised of one or more aqueous HBr absorption columns operating at 50° C. and 0.5 barg (50 kPag). The inlet stream 14 is contacted with a circulating aqueous HBr stream and HBr is absorbed. The absorber outlet stream, containing 65% wt. HBr, is pressurized, heated to 125° C., and flashed. The liquid stream from the flash (containing 20% wt HBr) is recycled to the absorber. The vapor HBr stream 23 is routed to the bromine generation reactor to recover bromine from HBr.

The organic stream 24 from the bromine recovery system 190 is routed to a conventional distillation column 200 that produces a light stream 25 comprised of primarily methane, ethane, ethylene, propane, and propylene and a heavy stream 26 comprised primarily of butanes, and pentanes. The light stream 25 is routed to a demethanizer column 240. The heavy stream 26 is fed to LO fractionation 250 (see below).

The portion of stream 9 that is not fed to the BTX coupling reactor 140 is heated to 400° C. and fed to the LO coupling reactor 210. In the LO coupling reactor, methyl bromide reacts adiabatically over a magnesium substituted ZSM-5 catalyst at 400° C. and 25 barg (2500 kPag) to produce a mixture of compounds comprised predominately of ethylene, propylene, butenes, pentenes, BTX, and brominated light hydrocarbons. The LO coupling reactor 210 is comprised of multiple fixed-bed catalytic reactors operating on a reaction/regeneration cycle. During the reaction phase, methyl bromide reacts to form mixed products. At the same time, coke is formed and gradually deactivates the catalyst.

During the regeneration phase of a reactor, reactor feed is redirected to reactors in the reaction phase of the cycle.

Heated air stream 47 is utilized to burn coke and regenerate the catalyst. Reactor effluent gas stream 48 from the decoking operation is routed to the bromine generation reactor to recover bromine.

The LO coupling reactor 210 effluent stream 28 is fractionated in a conventional distillation column 220 to produce stream 29, comprised primarily of butenes, pentenes, brominated light hydrocarbons, and BTX, and a light stream 30 comprised primarily of unreacted methane, ethylene, propylene, and HBR. The heavy stream 29 is recycled as feed to the BTX coupling reactor 140. The light stream is routed to a second HBr recovery system 230.

Process design for the HBr recovery system 230 is congruous with HBr recovery system 190, producing an HBr stream 31 that is routed to the bromine generation reactor 110 for recovery of bromine from HBr, and an organic stream 32 that is routed to the demethanizer 240.

Stream 32 from the HBr recovery system 230 and stream 25 from distillation column 200 are fed to the demethanizer 240, which produces a light stream 2 comprised of primarily methane, which is recycled to the bromination reactor 100. The demethanizer 240 also produces a heavy stream 33, comprised of ethane, ethylene, propane, and propylene, which is fed to a light olefin fractionation unit 250.

The light olefin fraction unit 250 utilizes conventional distillation to produce ethane stream 34, which is sold as a product or used as fuel, product ethylene stream 35, product propylene stream 36, and LPG stream 37. The ethane stream 34 and LPG stream 37 could alternately be used steam cracker feed.

Hydrogen bromide in line 23 from HBr recovery 190, and in line 31 from HBr recovery 230 is heated and fed to the bromine generation reactor 110. Cupric oxide catalyst is used to convert HBr to bromine and water. Air is compressed and fed through line 39 to bromine generation reactor 110 and the catalyst is continuously regenerated. The light ends stream 17 generated in distillation column 170 and regeneration gas from the bromination reactor 100 (line 44), BTX coupling reactor 140 (line 46), and the LO coupling reactor 210 (line 48) are also fed to bromine generation reactor 110.

Bromine generation reactor 110 is comprised of several shell/tube exchangers whose tubes are filled with copper oxide catalyst. Heat released by the exothermic conversion of hydrogen bromide to bromine and water is removed by generation of steam (line 41) and may be subsequently utilized elsewhere in the process (e.g. in the fractionation steps). The effluent from bromination reactor 110 is further cooled, generating additional steam. The inert gases, primarily nitrogen and unreacted oxygen are routed to a bromine scavenging adsorbent (not shown) and then released through line 42. The liquid product from bromine generation reactor 110, comprised of water and bromine, is phase separated at sub-ambient temperature and then distilled to produce a water stream 40 and a bromine stream 5 which is dried and recycled to bromination reactor 100. The water stream 40 is further purified and released.

### EXAMPLE 1

Referring to FIG. 1, the flow rate in methane feed line 1 to the bromination reactor 100 is 100 kg/hr. Reactor 100 is operated at 510° C. and 3000 kPag. The conversion of methane is 50% and the selectivity to methyl bromide is 67%.

Reproportionation reactor 120 is also operated at 510° C. and 3000 kPag. The conversion is 43% and the selectivity to methyl bromide is 100%.

BTX coupling reactor 140 is operated at 425° C. and 2500 kPag. The conversion of methyl bromide is 100% and the selectivity to BTX is 32%.

LO coupling reactor 210 is operated at 400° C. and 2500 kPag. The conversion of methyl bromide is 100% with a selectivity to light olefins (ethane and propylene) of 55%.

Bromine generation reactor 110 is operated at 375° C. and 200 kPag and the flow rate of air through feed line 36 is 554 kg/hr. The conversion and selectivity are both 100%. The flow rate in water stream 42 is 111 kg/hr.

The process produces 4 kg/hr of ethane, 7 kg/hr of LPG, 9 kg/hr of benzene, 14 kg/hr of p-xylene, 17 kg/hr of ethylene and 29 kg/hr of propylene.

What is claimed is:

1. An integrated process for producing aromatic hydrocarbons and ethylene and/or propylene from low molecular weight alkanes, preferably methane, which comprises:
(a) contacting one or more low molecular weight alkanes, preferably methane, with a halogen, preferably bromine, under process conditions sufficient to produce a monohaloalkane, preferably monobromomethane,
(b) reacting a first portion of the monohaloalkane in the presence of a coupling catalyst under process conditions sufficient to produce aromatic hydrocarbons and C₂₋₅ alkanes and optionally C₂₋₅ alkenes,
(c) separating the aromatic hydrocarbons from the product mixture of step (b) to produce aromatic hydrocarbons, and
(d) reacting a second portion of the monohaloalkane in the presence of a coupling catalyst under process conditions sufficient to produce ethylene and propylene.

2. The process of claim 1 wherein C₂ alkanes and alkenes are separated from at least part of the C₂-C₅ alkanes and alkenes produced in step (b) and at least part of the remaining C₃₋₅ alkanes and alkenes are liquefied to produce LPG.

3. The process of claim 1 wherein at least part of the alkyl-bromides produced in step (d) are recycled to step (b).

4. The process of claim 1 wherein C₄₊ alkanes and alkenes are separated from the other alkanes and alkenes and, together with alkylbromide byproducts produced in step (d), are recycled to step (b).

5. The process of claim 1 wherein ethylene and/or propylene are produced in step (d) and at least part of the ethylene and/or propylene is recycled to step (b).

6. The process of claim 1 wherein at least some unconverted methane and/or at least some of any produced methane is recovered and recycled to step (a).

7. The process of claim 1 wherein ethane and/or propane is produced in step (b) and/or (c) and at least some of the ethane and/or propane is recycled to step (a).

8. The process of claim 1 wherein multi-brominated methane species are produced in step (a) and are separated from the monobromomethane prior to steps (b) and (d) and recycled to step (a).

9. The process of claim 1 wherein hydrogen bromide is produced and at least some of said hydrogen bromide is converted to bromine which is recycled to step (a).

10. The process of claim 1 wherein hydrogen bromide is produced in the bromination step and removed prior to the coupling step.

11. The process of claim 1 wherein at least some hydrogen bromide is present in the C₂₋₅ alkanes stream and is removed therefrom prior to liquefaction to LPG.

12. The process of claim 1 wherein aromatic C₉₊ hydrocarbons are also produced in step (b) and are separated from the other products of step (b) and transalkylated to xylenes with toluene and/or hydrodealkylated to produce benzene, toluene and xylenes.

13. The process of claim 1 wherein multi-halogenated species are formed in step (a) and are reproportionated to form more monohaloalkane.

14. The process of claim 1 wherein the low molecular weight alkanes are comprised of methane and the halogen is bromine.

15. The process of claim 1 wherein at least part of the energy released in the conversion of hydrogen bromide to bromine is recovered and utilized in steps (a)-(c) or any combination thereof and optionally in upstream and/or downstream processing.

16. The process of claim 1 wherein the aromatic hydrocarbons comprise at least in part a xylene mixture and para-xylene is produced by the steps of 1) recovering para-xylene from the xylene mixture, 2) re-isomerizing the para-xylene-deprived xylene mixture to an equilibrium mixture, and 3) repeating steps 1) and 2).

17. A process for producing phenol which comprises producing benzene by
(a) contacting one or more low molecular weight alkanes, preferably methane, with a halogen, preferably bromine, under process conditions sufficient to produce a monohaloalkane, preferably monobromomethane,
(b) reacting a first portion of the monohaloalkane in the presence of a coupling catalyst under process conditions sufficient to produce aromatic hydrocarbons and C₂₋₅ alkanes and optionally C₂₋₅ alkenes,
(c) separating the aromatic hydrocarbons from the product mixture of step (b) to produce aromatic hydrocarbons, and
(d) reacting a second portion of the monohaloalkane in the presence of a coupling catalyst under process conditions sufficient to produce ethylene and propylene;
and then either:
1) reacting benzene with propylene to produce cumene, oxidizing the cumene to produce cumene hydroperoxide and then hydrolyzing the cumene hydroperoxide in an acidic medium to produce phenol, or
2) directly oxidizing benzene using air or oxygen, or
3) sulfonating the benzene and then hydrolyzing the sulfonate product, or
4) chlorinating the benzene and the hydrolyzing the chlorinated product to produce phenol.

18. The process of claim 17 wherein the propylene used to produce cumene is a mixture of propylene and propane produced in step (d).

19. A process for producing styrene which comprises producing benzene by
(a) contacting one or more low molecular weight alkanes, preferably methane, with a halogen, preferably bromine, under process conditions sufficient to produce a monohaloalkane, preferably monobromomethane,
(b) reacting a first portion of the monohaloalkane in the presence of a coupling catalyst under process conditions sufficient to produce aromatic hydrocarbons and C₂₋₅ alkanes and optionally C₂₋₅ alkenes,
(c) separating the aromatic hydrocarbons from the product mixture of step (b) to produce aromatic hydrocarbons, and
(d) reacting a second portion of the monohaloalkane in the presence of a coupling catalyst under process conditions sufficient to produce ethylene and propylene; and then reacting the benzene with ethylene to produce ethylbenzene and then dehydrogenating the ethylbenzene to produce styrene.

20. A process for producing terephthalic acid which comprises producing paraxylene by
(a) contacting one or more low molecular weight alkanes, preferably methane, with a halogen, preferably bromine, under process conditions sufficient to produce a monohaloalkane, preferably monobromomethane,
(b) reacting a first portion of the monohaloalkane in the presence of a coupling catalyst under process conditions sufficient to produce aromatic hydrocarbons and C₂₋₅ alkanes and optionally C₂₋₅ alkenes,
(c) separating the aromatic hydrocarbons from the product mixture of step (b) to produce aromatic hydrocarbons, and
(d) reacting a second portion of the monohaloalkane in the presence of a coupling catalyst under process conditions sufficient to produce ethylene and propylene;
and then oxidizing the paraxylene in the presence of molecular oxygen to produce terephthalic acid.
